(19) United States
(12) Patent Application Publication
(10) Pub. No.: US 2004/0172069 A1
(43) Pub. Date: Sep. 2, 2004

(54) Recording information for emergency call by defibrillator apparatus

(76) Inventor: Douglas T. Hakala, Woodinville, WA (US)

Correspondence Address: SHUMAKER & SIEFFERT, P. A., 8425 SEASONS PARKWAY, SUITE 105, ST. PAUL, MN, 55125, US

(21) Appl. No.: 10/377,991

(22) Filed: Feb. 28, 2003

(51) Int. Cl. A61N 1/39

(52) U.S. Cl. 607/5

(57) ABSTRACT

Techniques for recording information for an emergency call to be placed by a defibrillator apparatus are disclosed. The defibrillator apparatus includes a defibrillator, and may also include a defibrillator base. A user may enter information, such as the location where the defibrillator apparatus is installed, which may be stored in a memory of the defibrillator apparatus. The defibrillator apparatus may prompt the user to enter the information. The defibrillator apparatus may receive words spoken by the user, and store the spoken words as a voice recording that includes the information. The defibrillator apparatus may provide the information, which may include a voice recording, to an emergency response center, for example, in response to receiving an indication that the defibrillator is being deployed to treat a patient.

### TECHNICAL FIELD

The invention relates to defibrillators and, more particularly, to automatic and semi-automatic external defibrillators.

### BACKGROUND

Ventricular fibrillation is characterized by rapid, irregular, uncoordinated depolarizations of the ventricles of a heart. Ventricular fibrillation is particularly life threatening because activity within the ventricles of the heart is so uncoordinated that virtually no pumping of blood takes place. If untreated, a patient whose heart is undergoing fibrillation may die within a matter of minutes.

An external defibrillator may be used to deliver an electrical pulse to a fibrillating heart. The pulse may depolarize the heart and cause it to reestablish a normal sinus rhythm. Increasingly, in order to reduce the amount of time between onset of ventricular fibrillation and treatment, automatic or semi-automatic external defibrillators are installed in areas such as sports venues, concert halls, museums, golf courses, shopping malls, restaurants, places of employment and the like. Such defibrillators are located and configured such that an untrained layperson or minimally trained first responder may use them to provide prompt defibrillation therapy to a patient experiencing ventricular fibrillation.

It is also important, however, that an emergency response center be promptly contacted so that paramedics, or the like, may be sent to more completely treat the patient. The user of the defibrillator may take time to locate a phone and call the emergency response center before providing defibrillation therapy, may make the call after providing defibrillation therapy, or a bystander may be recruited to make the call. The arrival of paramedics, the delivery of therapy, or both may be delayed. Moreover, whoever calls the emergency response center may not be able to adequately describe the location of the patient, and any display at the emergency response center of the location of the phone used to call the emergency response center may not correlate with the location of the patient, further delaying the arrival of paramedics.

### SUMMARY

In general, the invention is directed to techniques for recording information for an emergency call to be placed by a defibrillator apparatus. The defibrillator apparatus includes a defibrillator, and may also include a defibrillator base. The defibrillator base may provide support to the defibrillator, allowing it to be mounted on a wall. In some embodiments, either the defibrillator or the defibrillator base may record emergency call information and make the emergency call.

A user may enter information, such as the location where the defibrillator apparatus is installed, which may be stored in a memory of the defibrillator apparatus, e.g., upon installation at a particular location. The defibrillator apparatus may prompt a user, such as an install technician, to enter the information. The defibrillator apparatus may receive words spoken by the user, and store the spoken words as a voice recording that includes the information.

The defibrillator apparatus may provide the recorded information, which may include a voice recording, to an emergency response center. The defibrillator apparatus may provide the information, for example, in response to receiving an indication that the defibrillator is being deployed for use to treat a patient. Deployment may be indicated by removal of the defibrillator from the base, defibrillator power-on, attachment of an electrode set coupled to the defibrillator to a patient, or other events indicative of use.

In one embodiment, the invention is directed to a method in which information is communicated from a defibrillator apparatus to an emergency response center. A pre-recorded voice message is included in the communicated information. The voice message may include information identifying an installation site for the defibrillator apparatus.

In another embodiment, the invention is directed to a device that includes a memory and a processor. The memory stores a pre-recorded voice message. The processor communicates information from a defibrillator apparatus to an emergency response center that includes the pre-recorded voice message. The voice message may include information identifying an installation site for the defibrillator apparatus.

In another embodiment, the invention is directed to a method in which words spoken by a user that include emergency call information are received. At least some of the spoken words are stored in a memory as a voice message that includes the emergency call information. The method may include prompting the user to provide a vocal response that includes the emergency call information. Words that include a description of a location of the defibrillator apparatus may be received, and the voice message may include the description.

In another embodiment, the invention is directed to a device that includes a microphone, a processor, and a memory. The microphone detects words spoken by a user. The processor receives words spoken by the user that include emergency call information via the microphone. The memory stores a voice message that includes the emergency call information. The device may also include a speaker to provide audio prompts to the user. The processor may prompt the user to provide a vocal response that includes the emergency call information via the speaker. The spoken words received by the processor may include a description of a location of a defibrillator apparatus, and the voice message may include the description.

In another embodiment, the invention is directed to a system that includes a defibrillator and a defibrillator base. The defibrillator provides defibrillation therapy to a patient. The defibrillator base receives an indication that the defibrillator is being deployed, and communicates information to an emergency response center that includes a pre-recorded voice message. The voice message may include information identifying an installation site for the system.

In another embodiment, the invention is directed to a method in which a defibrillator apparatus is installed at a location. Information about the location is entered in a memory of the apparatus.

The invention may provide one or more advantages. For example, by providing recorded information to an emergency response center, and particularly location information, a defibrillator apparatus may allow paramedics to more quickly respond to a medical emergency in which the defibrillator apparatus is used. The person using the defibrillator apparatus is allowed to more completely concentrate on treating the patient, rather than spend time and effort locating a telephone, placing a call, and conveying location information. If the recorded emergency call information includes location information, the response time for paramedics may be further improved.

Prompting a user for emergency call information may simplify the set-up process from the perspective of the user. Prompting the user for vocal responses may further simplify the set-up process. All or some of the information provided by the user then may be communicated to an emergency response center when the defibrillator apparatus is deployed. By providing emergency call information to an emergency response center in the form a voice recording, a defibrillator apparatus may be made more compatible with existing emergency response centers that are only capable of processing voice calls.

The details of one or more embodiments of the invention are set forth in the accompanying drawings and the description below. Other features, objects, and advantages of the invention will be apparent from the description and drawings, and from the claims.

### BRIEF DESCRIPTION OF DRAWINGS

FIG. 1 is a block diagram illustrating an environment in which a defibrillator apparatus according to the invention may make an emergency call to an emergency response center.

FIG. 2 is a perspective diagram illustrating an example defibrillator apparatus according to an embodiment of the invention.

FIG. 3 is a block diagram illustrating an example defibrillator base according to an embodiment of the invention.

FIG. 4 is a block diagram illustrating an example defibrillator according to an embodiment of the invention.

FIG. 5 is a flowchart illustrating an example method that may be employed by a defibrillator apparatus to receive emergency call information according to an embodiment of the invention.

FIG. 6 is a flowchart illustrating an example method that may be employed by a defibrillator apparatus to make an emergency call according to an embodiment of the invention.

### DETAILED DESCRIPTION

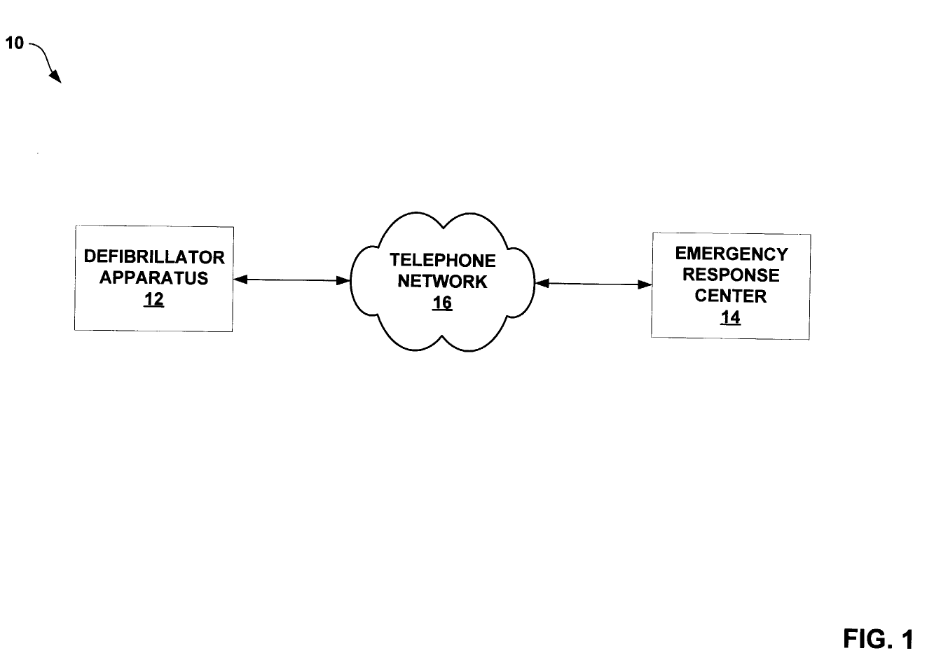

FIG. 1 is a block diagram illustrating an environment 10 in which a defibrillator apparatus 12 according to the invention may make an emergency call to an emergency response center 14. Defibrillator apparatus 12 makes the emergency call in order to provide information, such as the location of the defibrillator apparatus, to emergency response center 14. Emergency response center 14 may be a dispatch center for an emergency medical system (EMS) and, by providing information to emergency response center 14, defibrillator apparatus 12 may allow paramedics to more quickly respond to a medical emergency in which defibrillator apparatus 12 is used. Defibrillator apparatus 12 may make the emergency call and provide the information to emergency response center 14 via a telephone network 16, which may include a public switched telephone network (PSTN) and/or cellular telephone network.

A user (not shown) may enter the emergency call information using defibrillator apparatus 12, and defibrillator apparatus 12 stores the emergency call information. The user may, for example, install defibrillator apparatus 12 at a location, and enter the emergency call information, including information concerning the location of the defibrillator apparatus 12, during installation. In some embodiments, defibrillator apparatus 12 prompts a user, such as an install technician, to enter the emergency call information. Defibrillator apparatus 12 may provide the user with a series of prompts that guide the user through the process of entering the emergency call information, simplifying the set-up process from the perspective of the user.

In some embodiments of defibrillator apparatus 12, the user may verbally enter emergency call information. Defibrillator apparatus 12 may store a voice recording of the words uttered by the user, and play the voice recording for emergency response center 14. Such embodiments of defibrillator apparatus 12 may further simplify the set-up process from the perspective of the user, and may be able to provide emergency call information to existing emergency response centers 14 that are only capable of processing voice calls, e.g., emergency response centers 14 that rely on an operator to answer a phone and dispatch paramedics.

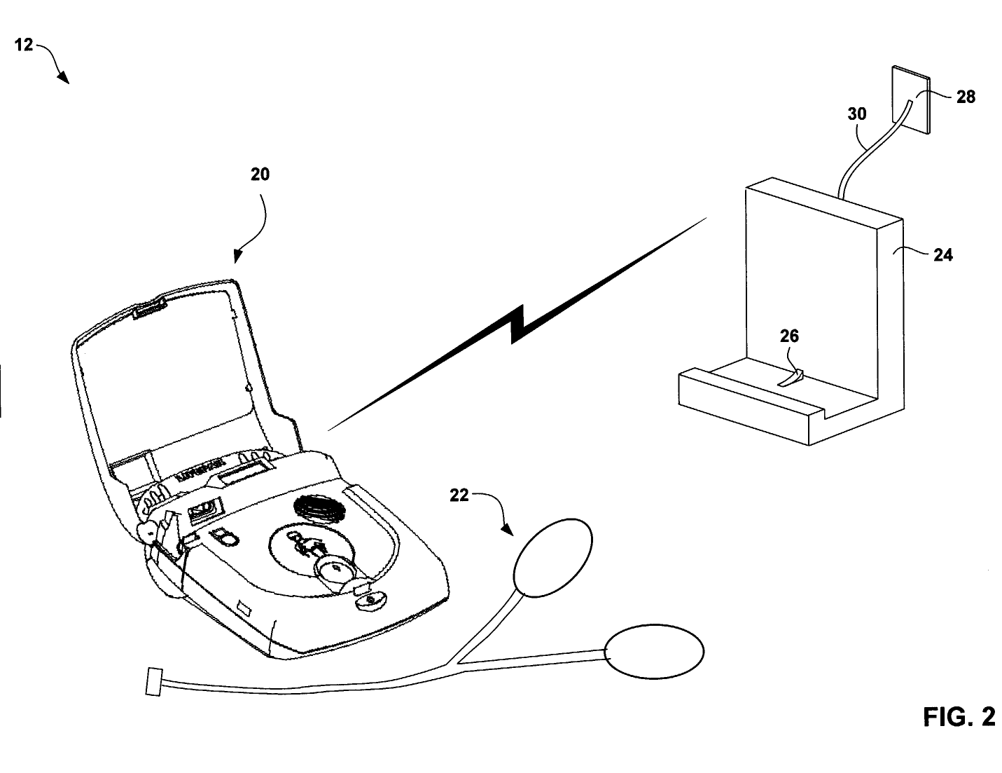

FIG. 2 is a perspective diagram illustrating components of defibrillator apparatus 12 according to an embodiment of the invention. Defibrillator apparatus 12 includes a defibrillator 20. Defibrillator 20 may be used to deliver therapy in the form of electrical pulses to a patient whose heart is undergoing ventricular defibrillation via electrode set 22. Defibrillator 20 may be an automatic or semi-automatic external defibrillator.

Defibrillator apparatus 12 may also include a defibrillator base 24. Defibrillator base 24 may hold or otherwise physically interact with defibrillator 20 when defibrillator 20 is not in use. Defibrillator base 24 may, as depicted in FIG. 2, provide support for defibrillator 20 such that defibrillator apparatus 12 may be mounted as a unit on a wall, or the like.

However, the configuration of defibrillator base 24 illustrated in FIG. 2 is merely exemplary. For example, in some embodiments, defibrillator base 24 may include a case with a door or breakable glass pane to allow access to defibrillator 20. In other embodiments, defibrillator apparatus 12 may not be mounted on a vertical structure. In still other embodiments, defibrillator base 24 may not physically interact with defibrillator 20 at all. Instead, in some embodiments, defibrillator base 24 may provide a communication link for defibrillator 20.

Base 24 may make a call to emergency response center 14 in response to receiving an indication that defibrillator 20 is being deployed to treat a patient. A user may remove defibrillator 20 from base 24 to treat a patient. Base 24 may detect that defibrillator 20 has been removed from base 24 via hook-switch 26, which is depressed when defibrillator 20 is being supported by base 24. The invention is not, however, limited to detection of removal of defibrillator 20 via hook-switch 26. For example, both defibrillator 20 and base 24 may include a variety of detection mechanisms, such as electrical contacts, optical or magnetic proximity sensors, and the like. In each case, base 24 may detect removal of defibrillator 20 and automatically initiate a call to emergency response center 14.

Moreover, base 24 need not detect deployment of defibrillator 20 by detecting removal of defibrillator 20. For example, in some embodiments, base 24 may receive an indication that defibrillator is being deployed via wireless communication with defibrillator 20. Wireless communication between base 24 and defibrillator 20 may be radio frequency communication, and may be according to any of a number of local wireless communication standards, such as the 802.11 or Bluetooth specification sets. Defibrillator 20 may, for example, send a message indicating deployment upon powering or detecting that electrode set 22 has been attached to a patient. By detecting deployment in this manner, base 24 may avoid calling emergency response station 14 in situations where defibrillator 20 is removed from base 24 for reasons other than to treat a patient, e.g., for maintenance or inspection.

In order to make a call to emergency response center 14, base 24 may interface with telephone network 16 via a telephone jack 28 and cable 30. In other embodiments, base 24 may interface with telephone network 16 via a cellular telephone transceiver (not shown). Such embodiments of base 24 do not require the presence of telephone jack 28 for installation, and may therefore be installed in a broader range of locations.

Defibrillator apparatus 12 is not limited to embodiments in which base 24 makes emergency calls. Rather, defibrillator 20 may include a cellular transceiver (not shown) to call and provide emergency call information to emergency response center 14. Defibrillator 20 may include switch-hook 26, or the like, and may detect deployment by detecting removal from base 24. As further examples, defibrillator 20 may alternatively detect deployment based on powering on, or by detecting attachment of electrode set 22 to a patient. In response, defibrillator 20 itself may initiate a telephone call to emergency response center 14. Either defibrillator 20 or base 24 may be configured to receive emergency call information from a user during set-up. Accordingly, in some embodiments, defibrillator apparatus 12 may not include base 24 at all.

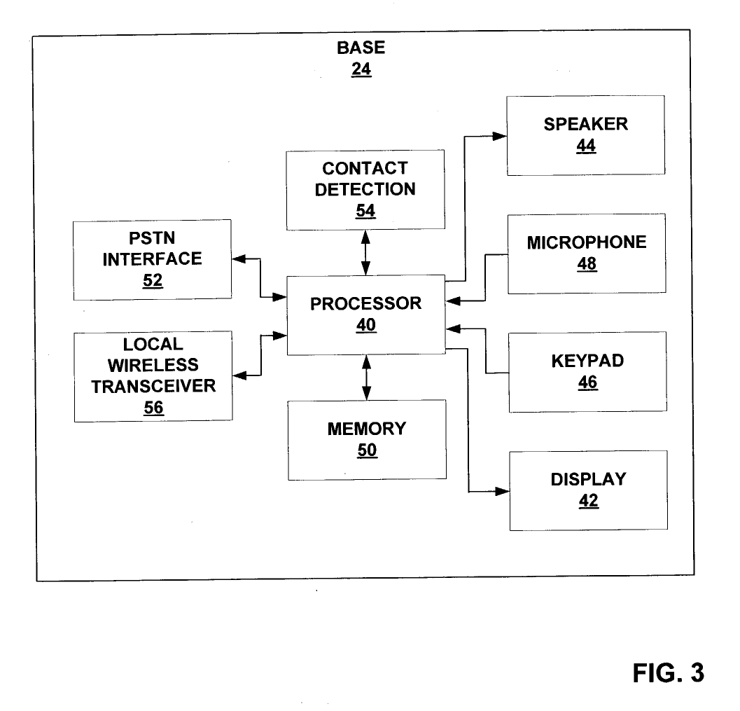

FIG. 3 is a block diagram illustrating an example configuration of defibrillator base 24 according to an embodiment of the invention. Base 24 includes a processor 40, which controls base 24 to function as described herein. Processor 40 may include a microprocessor, ASIC, DSP, FPGA, discrete logic circuitry, or the like.

Base 24 may include a display 42, such as a LED or LCD display, and/or a speaker 44 for communicating information to a user. Processor 40 may prompt a user to provide emergency call information during set-up via one or both of display 42 and speaker 44. Base 24 may also include a keypad 46, which may be an alphanumeric keypad, which a user may use to enter emergency call information during set-up.

Base 24 may additionally or alternatively include a microphone 48 to detect words spoken by a user. Processor 40 may receive words spoken by a user that include emergency call information via microphone 48. The emergency call information may include location information or other information useful in identifying the general location of the defibrillator 20, and hence the location of a patient. Processor 40 may store emergency call information received from a user during set-up, which may include an audio recording containing some or all of the spoken words, in a memory 50. Memory 50 may include any of a variety of solid state, magnetic or optical media, such as RAM, ROM, CD-ROM, magnetic disk, or EEPROM.

In the example of FIG. 3, base 24 may include a PSTN interface circuit 52 to interface with telephone network 16. Processor 40 may control PSTN interface circuit 52 to call emergency response center 14, and may provide emergency call information to emergency response center 14 via interface circuit 52. Alternatively, base 24 may include a cellular transceiver (not shown), and processor may call emergency response center 14, and provide emergency call information to emergency response center 14 via the cellular transceiver.

Processor 40 may detect deployment of defibrillator 20 by detecting removal of defibrillator 20 from base 40 via a contact detection circuit 54, which may include hook-switch 26. Processor 40 may additionally or alternatively receive an indication of deployment from defibrillator 20 via wireless communication with defibrillator 20 as described above. Base 24 may include a local wireless transceiver 56 for wireless communications with defibrillator 20. Transceiver 56 may include an antenna to facilitate wireless communication via a radio frequency communication medium, and may be compliant with one or more wireless communication standards such as to one or both of the 802.11 or Bluetooth specification sets.

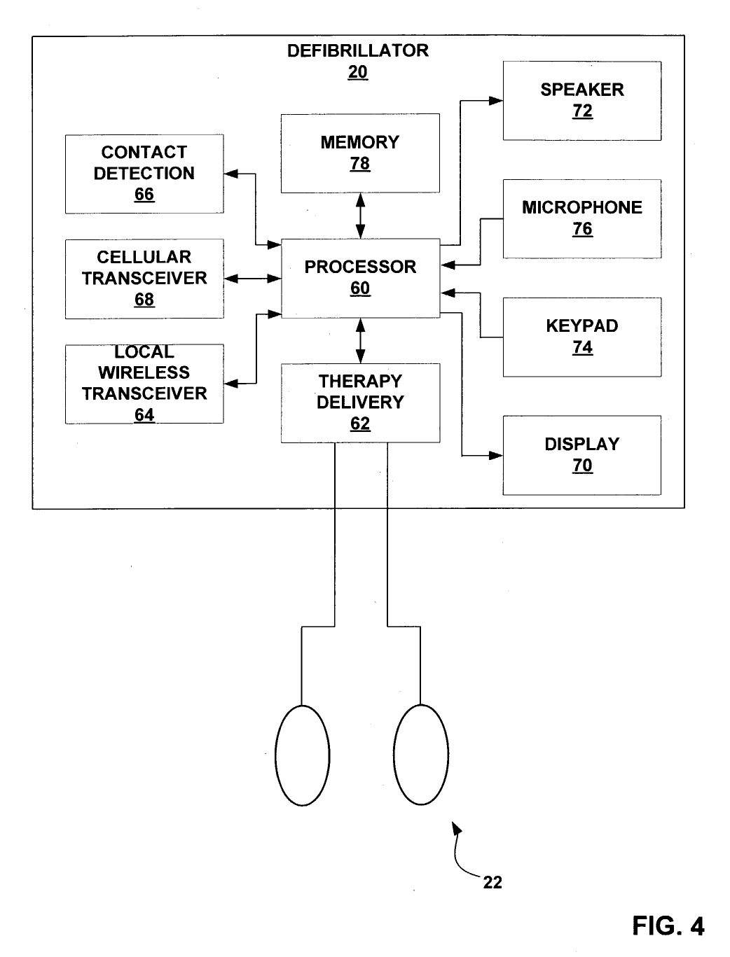

FIG. 4 is a block diagram illustrating an example configuration of defibrillator 20 according to an embodiment of the invention. Defibrillator 20 includes a processor 60, which controls defibrillator 20 to function as described herein. Processor 60 may include a microprocessor, ASIC, DSP, FPGA, discrete logic circuitry, or the like.

Defibrillator 20 includes a therapy delivery circuit 62, which may include circuits for the delivery of defibrillation pulses to a patient via electrode set 22. For example, therapy delivery circuit 62 may include an energy storage circuit, which may include capacitors, to store energy to deliver to a patient in the form of defibrillation pulses. Therapy delivery circuit 62 may also include a switch that, when activated, couples the energy storage circuit to electrode set 22. Processor 60 controls the delivery of therapy by therapy delivery circuit 62.

As described above, processor 60 may provide an indication of deployment of defibrillator 20 to base 24 via wireless communication with base 24. Defibrillator 20 may include a local wireless transceiver 64 for wireless communications with base 24. Transceiver 64 may include an antenna to facilitate wireless communication via a radio frequency communication medium, and may be compliant with one or more wireless communication standards such as to one or both of the 802.11 or Bluetooth specification sets.

Processor 60 may provide an indication of deployment of defibrillator 20 to base 24 upon power-up of defibrillator 20. Therapy delivery circuit 62 may detect attachment of electrode set 22 to a patient based on a change in the impedance between electrodes of electrode set 22. In this manner, processor 60 may provide an indication of deployment of defibrillator 20 to base 24 based on the detection by therapy delivery circuit. In some embodiments, defibrillator 20 may include a contact detection circuit 66, which may include hook-switch 26. Processor 60 may detect deployment of defibrillator 20 by detecting removal of defibrillator 20 from base 40 via a contact detection circuit 66, and may provide an indication of deployment of defibrillator 20 to base 24 based on the detection.

Defibrillator 20 or defibrillator base 24 may call and provide emergency call information to emergency response center 14. In the example of FIG. 4, defibrillator 20 may include a cellular transceiver 68, and processor 60 may control cellular transceiver 68 to call emergency response center 14. In this case, defibrillator 20 provides emergency call information to emergency response center 14 via cellular transceiver 68. Processor 60 may control cellular transceiver 68 to call emergency response center 14 in response to detecting deployment of defibrillator 20 by any of the methods described above.

Defibrillator 20 may receive and store emergency call information during set-up. Defibrillator 20 may include a display 70, such as an LED or LCD display, and/or a speaker 72 for communicating information to a user. Processor 60 may prompt a user to provide emergency call information during set-up via one or both of display 70 and speaker 72. Defibrillator 20 may also include a keypad 74, which may be an alphanumeric keypad, which a user may use to enter emergency call information during set-up. Defibrillator 20 may additionally or alternatively include a microphone 76 to detect words spoken by a user. Processor 60 may receive words spoken by a user that include emergency call information via microphone 76. Processor 60 may store emergency call information received from a user during set-up, which may include an audio recording, in a memory 78. Memory 78 may include any of a variety of solid state, magnetic or optical media, such as RAM, ROM, CD-ROM, magnetic disk, or EEPROM.

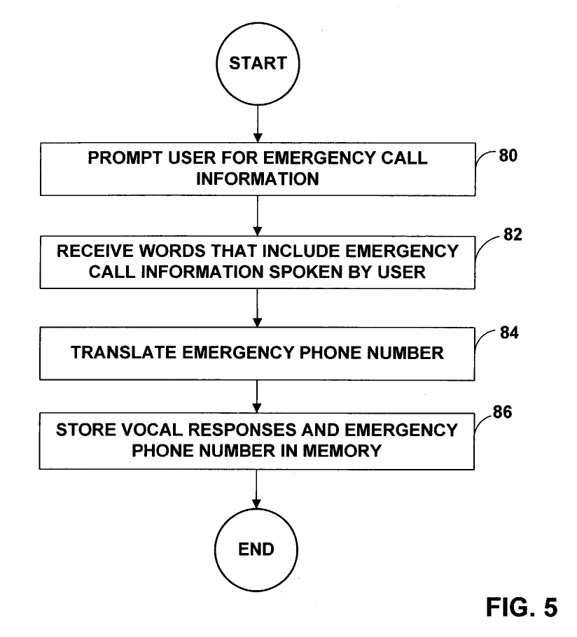

FIG. 5 is a flowchart illustrating an example method that may be employed by defibrillator apparatus 12 to receive emergency call information according to an embodiment of the invention. In particular, FIG. 5 illustrates an example method that may be employed by defibrillator apparatus 12 to receive emergency call information as words spoken by a user during set-up. Functions described as being performed by defibrillator apparatus 12 may be performed in various embodiments by either a defibrillator 20 or defibrillator base 24.

Defibrillator apparatus 12 may prompt the user for emergency call information (80). Defibrillator apparatus 12 may display the prompts via a display 42, 70, or provide audible prompts via a speaker 44, 72. Defibrillator apparatus 12 may provide a series of prompts that, in a structured way, direct the user to provide emergency call information. For example, in order to collect emergency call information relating to the installation location of defibrillator apparatus 12, defibrillator apparatus 12 may prompt the user for a name of the facility in which defibrillator apparatus 12 is installed, a street address of the facility, a description of the location of defibrillator apparatus 12 within the facility, and the phone number to dial in case of an emergency.

Defibrillator apparatus may then receive words spoken by the user that include the requested emergency call information via a microphone 48, 76 (82). For example, the user may verbally respond to each prompt provided by defibrillator apparatus 12 after each prompt is provided. Where defibrillator apparatus 12 provides the series of prompts requesting location information described above, the user may, for example, say “Metrodome” in response to the prompt for a facility name, say “900 South Fifth Street, Minneapolis” in response to the prompt for a facility address, say “second level concourse near entrance to section LL” in response to the prompt for a location description, and say “9-911” in response to the prompt for an emergency number. Defibrillator apparatus 12 may repeat or display each response to the user for confirmation.

Defibrillator apparatus 12 may use voice recognition and translation techniques known in the art or commercially available software to translate the response to the prompt for an emergency number into a digital representation of the telephone number (84). Alternatively, the user may enter the telephone number via a keypad 46, 74 in response to a prompt. Moreover, it is understood that in some embodiments, defibrillator apparatus 12 may receive all emergency call information via a keypad 46, 74. The vocal responses and the digital representation of the emergency telephone number may be stored in a memory 50,78.

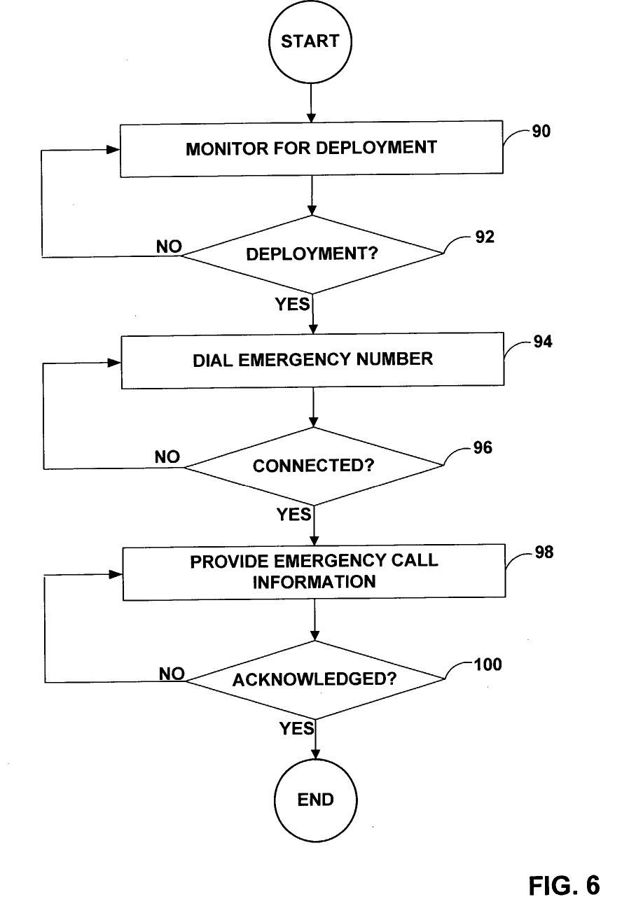

FIG. 6 is a flowchart illustrating an example method that may be employed by defibrillator apparatus 12 to make an emergency call according to an embodiment of the invention. Again, functions described as being performed by defibrillator apparatus 12 may be performed in various embodiments by either a defibrillator 20 or defibrillator base 24.

Defibrillator apparatus 12 may determine whether defibrillator 20 is being deployed by any of the method described above (90, 92). In response to determining that defibrillator 20 is being deployed, defibrillator apparatus 12 may dial an emergency number stored in a memory 50, 78 in order to call emergency response center 14 (94). A PSTN interface 52 or cellular transceiver 68 may repeatedly dial the emergency number until a connection with emergency response center 14 is established (96).

When a connection to emergency response center 14 is established, defibrillator apparatus 12 may provide emergency call information stored in a memory 50, 78 to emergency response center 14 (98). The emergency call information may be stored in the form of voice recordings, and defibrillator apparatus 12 may play the voice recording for emergency response center 14 via the connection. For example, where defibrillator apparatus 12 provided the example prompts discussed above during set-up, and received the example responses discussed above from the user during set-up, defibrillator apparatus may play a voice message for emergency response center 14: “This is an AED located at the Metrodome. There is a probable cardio-respiratory emergency in progress. The address is 900 South Fifth Street, Minneapolis. The location is second level concourse near entrance to section LL.” The italics indicate the voice recordings stored by defibrillator apparatus 12.

The voice message may be repeated until acknowledged by emergency response center 14 (100). For example, an operator at emergency response center 14 may enter an acknowledgment code via a touch-tone phone interface at emergency response center 14 when the message is received and understood. The code may be a simple combination of keys, such as *7.

Various embodiments of the invention have been described. However, one skilled in the art will appreciate that various additions or modifications may be made to these embodiments without departing from the scope of the invention. For example, after defibrillator apparatus 12 establishes a connection with and provides emergency call information to emergency response center 14, defibrillator apparatus 12 may open a voice channel between the user who is treating a patient with defibrillator 20 via a speaker 72 and microphone 76 of defibrillator 20.

This voice channel may, for example, allow emergency response center 14 to ask the user for more detailed information concerning the location or condition of the patient. The voice channel may also allow emergency response center 14 to coach the user on proper treatment of the patient, such as proper use of defibrillator 20 or proper CPR techniques. In embodiments of defibrillator apparatus 12 that include a base 24 that calls emergency response center 14, the voice channel may be provided via wireless communication between defibrillator 20 and base 24. These and other embodiments are within the scope of the following claims.

1. A method comprising:
communicating information from a defibrillator apparatus to an emergency response center; and
including a pre-recorded voice message in the communicated information.

2. The method of claim 1, in which the voice message includes information identifying an installation site for the defibrillator apparatus.

3. The method of claim 1, further comprising retrieving the voice message from a memory, wherein the voice message includes speech uttered by a person following installation of the defibrillator apparatus.

4. The method of claim 1, in which communicating information comprises:
receiving an indication that a defibrillator of the defibrillator apparatus is being deployed; and
communicating information in response to the indication.

5. The method of claim 4, in which receiving an indication that a defibrillator is being deployed comprises detecting removal of the defibrillator from a defibrillator base of the defibrillator apparatus.

6. The method of claim 4, in which receiving an indication that a defibrillator is being deployed comprises receiving at least one of an indication that the defibrillator has been powered on and an indication that electrodes of the defibrillator have been attached to a patient.

7. The method of claim 4, in which receiving an indication that a defibrillator is being deployed comprises receiving the indication via a local wireless communication session with the defibrillator.

8. The method of claim 1, in which communicating information comprises establishing telephonic connection with the emergency response center via at least one of a public switched telephone network and a cellular telephone network.

9. The method of claim 1, in which communicating information comprises repeating the voice message until an acknowledgment is received from the emergency response center.

10. The method of claim 1, in which communicating information comprises communicating information from at least one of a defibrillator and a defibrillator base of the defibrillator apparatus.

11. A device comprising:
a memory to store a pre-recorded voice message; and
a processor to communicate information from a defibrillator apparatus to an emergency response center that includes the pre-recorded voice message.

12. The device of claim 11, in which the voice message includes information identifying an installation site for the defibrillator apparatus.

13. The device of claim 11, in which the voice message includes speech uttered by a person following installation of the defibrillator system.

14. The device of claim 11, in which the processor receives an indication that a defibrillator of the defibrillator apparatus is being deployed, and communicates information in response to the indication.

15. The device of claim 14, further comprising a detection circuit to detect removal of the defibrillator from a defibrillator base of the defibrillator apparatus, wherein the processor receives the indication that the defibrillator is being deployed by receiving an indication that the defibrillator has been removed from the base via the detection circuit.

16. The device of claim 14, in which the indication includes at least one of an indication that the defibrillator has been powered on and an indication that electrodes of the defibrillator have been attached to a patient.

17. The device of claim 14, further comprising a transceiver to establish a local wireless communication session with the defibrillator, wherein the processor receives the indication that the defibrillator is being activated via the local wireless communication session.

18. The device of claim 11, further comprising at least one of a public switched telephone network interface to establish a telephonic connection with the emergency response center via a public switched telephone network and a cellular telephone transceiver to establishing a telephonic connection with the emergency response center via a cellular telephone network, wherein the processor communicates information via the telephonic connection.

19. The device of claim 11, in which the processor repeats the voice message until the processor receives an acknowledgment from the emergency response center.

20. The device of claim 11, in which the device comprises one of a defibrillator and a defibrillator base of the defibrillator apparatus.

21. The device of claim 20, in which the defibrillator comprises an automatic external defibrillator.

22. A method comprising:
receiving words spoken by a user that include emergency call information; and
storing at least some of the spoken words in a memory as a voice message that includes the emergency call information.

23. The method of claim 22, further comprising prompting the user to provide a vocal response that includes the emergency call information.

24. The method of claim 22, in which receiving words spoken by a user comprises receiving words that include a description of a location of a defibrillator apparatus, and the voice message includes the description.

25. The method of claim 22, further comprising:
communicating information from a defibrillator apparatus to an emergency response center; and
including the pre-recorded voice message in the communicated information.

26. The method of claim 25, in which receiving words spoken by a user comprises receiving words that include a telephone number for an emergency response center, the method further comprising:
translating the spoken words that include the telephone number into a digital representation of the telephone number; and
storing the digital representation in the memory.

27. The method of claim 26, in which communicating information comprises establishing a telephonic connection with the emergency response center by dialing the telephone number using the digital representation.

28. A device comprising:
a microphone to detect words spoken by a user;
a processor to receive words spoken by the user that include emergency call information via the microphone; and
a memory to store a voice message that includes the emergency call information.

29. The device of claim 28, further comprising a speaker to provide audio prompts to the user, wherein the processor prompts the user to provide a vocal response that includes the emergency call information via the speaker.

30. The device of claim 28, in which the processor receives spoken words from the user that include a description of a location of a defibrillator apparatus, and the voice message includes the description.

31. The device of claim 28, in which the processor communicates information from a defibrillator apparatus to an emergency response center that includes the voice message.

32. The device of claim 31, in which the processor receives spoken words from the user that include a telephone number for the emergency response center, translates the spoken words that include the telephone number into a digital representation of the telephone number, and stores the digital representation in the memory.

33. The device of claim 32, further comprising a telephonic communications circuit, wherein the processor controls the telephonic communications circuit to establish a telephonic connection with the emergency response center by controlling the telephonic communications circuit to dial the telephone number using the digital representation.

34. A system comprising:
a defibrillator to provide defibrillation therapy to a patient; and
a defibrillator base to receive an indication that the defibrillator is being deployed, and communicate information to an emergency response center that includes a pre-recorded voice message.

35. The system of claim 34, in which the voice message includes information identifying an installation site for the system.

36. The system of claim 34, in which the voice message includes speech uttered by a person following installation of the system.

37. The system of claim 34, in which the defibrillator base includes a detection circuit, and receives an indication that the defibrillator is being deployed by detecting removal of the defibrillator from the defibrillator base via the contact detection circuit.

38. The system of claim 34, in which the defibrillator and the defibrillator base include local wireless communications transceivers and establish a local wireless communication session, and the defibrillator base receives the indication by receiving at least one of an indication that the defibrillator has been powered on and an indication that electrodes of the defibrillator have been attached to a patient from the defibrillator via the local wireless communication session.

39. The system of claim 34, in which the defibrillator base includes a microphone and a memory, receives words spoken by a user that include emergency call information via the microphone, and stores at least some of the spoken words in a memory as the voice message.

40. The system of claim 34, in which the defibrillator base includes a speaker, and prompts the user to provide vocal responses that include emergency call information via the speaker.

41. A method comprising:
installing a defibrillator apparatus at a location; and
entering information about the location in a memory of the apparatus.

42. The method of claim 41, in which the apparatus includes a defibrillator and a base.

43. The method of claim 41, in which entering is by speaking into the apparatus.

44. The method of claim 41, in which entering is by pressing keys.

45. The method of claim 41, in which entering is responsive to a prompt.

46. The method of claim 41, further comprising communicating the entered information to an emergency response center.

47. The method of claim 46, in which communicating comprises reproducing a voice spoken into the apparatus.

48. The method of claim 46, in which communicating comprises:
receiving an indication that a defibrillator of the defibrillator apparatus is being deployed; and
communicating information in response to the indication.

49. The method of claim 46, in which communicating comprises establishing telephonic connection with the emergency response center via at least one of a public switched telephone network and a cellular telephone network.
